(19) United States
(12) Patent Application Publication
(10) Pub. No.: US 2015/0087682 A1
(43) Pub. Date: Mar. 26, 2015

(54) Method for Treating Scleroderma

(71) Applicant: The Regents of the University of Colorado, A body corporate, Denver, CO (US)

(72) Inventors: Hang Yin, Boulder, CO (US); John Varga, Chicago, IL (US); Swati Bhattacharyya, Oakpark, IL (US)

(73) Assignee: The Regents of the University of Colorado, a body Corporate, Denver, CO (US)

(21) Appl. No.: 14/388,990

(22) PCT Filed: Apr. 17, 2013
(86) PCT No.: PCT/US13/36866

§ 371 Date: Sep. 29, 2014

Related U.S. Application Data
(60) Provisional application No. 61/625,660, filed on Apr. 17, 2012.

(51) Int. Cl. A61K 31/415

(52) U.S. Cl. CPC A61K 31/415
USPC 514/406

(57) ABSTRACT

The present invention provides a method for treating scleroderma by administering a therapeutically effective amount of a toll like receptor 4 inhibitor to a subject in need of such a treatment.

### CROSS-REFERENCE TO RELATED APPLICATIONS

This application claims the priority benefit of U.S. Provisional Application No. 61/625,660, filed Apr. 17, 2012, which is incorporated herein by reference in its entirety.

### STATEMENT REGARDING FEDERALLY FUNDED RESEARCH

This invention was made with government support under grant numbers NS067425 and AR043209 awarded by the National Institutes of Health. The government has certain rights in the invention.

### FIELD OF THE INVENTION

The present invention relates to a method for treating scleroderma. In particular, the present invention relates to treating scleroderma in a subject by administering a toll like receptor 4 (TLR4) inhibitor.

### BACKGROUND OF THE INVENTION

It is estimated that scleroderma or systemic sclerosis (SSc) affects 100,000-300,000 Americans, predominantly young to middle aged women. Systemic sclerosis is a progressive and untreatable disease of unknown cause and high mortality. Fibrosis in SSc resembles uncontrolled wound healing, where healing occurs by intractable fibrosis rather than normal tissue regeneration.

It is believed that SSc is associated with the highest case-fatality rates among the rheumatic diseases or connective tissue diseases. Currently, there are no validated biomarkers for diagnosis. Furthermore, no effective disease-modifying therapies are currently available. In fact, while some treatment can alleviate the pain associated with SSc, to date no therapy has been shown to significantly alter survival. The pathogenesis of SSc is characterized by early vascular injury, with inflammation followed by progressive tissue damage and fibrosis. Excessive production of collagen and ECM and accumulation of myofibroblasts in lesional tissues are believed to be responsible for progressive organ failure. Pathological fibrosis resembles a normal wound healing response that has become deregulated. It is estimated that fibrosis accounts for >25% of all deaths in the U.S. Thus, fibrosis represents one of the major unmet medical needs.

Accordingly, there is a need for an effective anti-fibrotic therapy.

### SUMMARY OF THE INVENTION

Some aspects of the invention provide methods for treating scleroderma or related autoimmune or a fibrotic condition in a subject by administering a therapeutically effective amount of a TLR4 inhibitor to the subject in need of such a treatment. In some embodiments, scleroderma is a systemic sclerosis, which is a systemic autoimmune disease or systemic connective tissue disease. SSc is often characterized by deposition of collagen in the skin. In some cases, SSc involves deposition of collagen in organs, such as the kidneys, heart, lungs and/or stomach.

In other embodiments, scleroderma is a diffuse scleroderma. Diffuse scleroderma typically affects the skin and organs such as the heart, lungs, gastrointestinal tract, and kidneys. Still in other embodiments, scleroderma is a limited scleroderma that affects primarily the skin including, but not limited to, that of the face, neck and distal elbows and knees. Still in other embodiments, scleroderma is a limited scleroderma. In some instances, the limited scleroderma include clinical conditions that affect the hands, arms, and face. In other instances, clinical conditions associated with the limited scleroderma include, calcinosis, Raynaud's phenomenon, esophageal dysfunction, sclerodactyl), telangiectasias and pulmonary arterial hypertension. Yet in other instances, scleroderma is a localized scleroderma.

Yet in other embodiments, a TLR4 inhibitor is a compound of the formula:

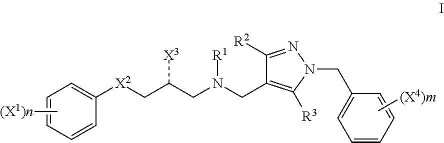

where
each of n and m is independently an integer from 0 to 5; typically each of m and n is independently an integer of 0-4; often each of m and n is independently an integer of 0-2;
each X¹ is independently alkoxide, optionally-substituted alkyl, or alkenyl;
X² is O, NR^{a}, or S;
X³ is —OR^{b}, —SR^{b}, or —NR^{b}R^{c};
each X⁴ is independently halide or alkoxide; and
each of R^{a}, R^{b}, R^{c}, R¹, R², and R³ is independently hydrogen or alkyl.

Within these embodiments, in some instances X² is O. Still in other instances, X³ is —OH. Yet in other instances, R¹, R² and R³ are alkyl. Typically, R¹, R², and R³ are methyl. Yet in other instances, X¹ is alkoxide, hetero-substituted alkyl or alkenyl-alkyl. Often X¹ is methoxide, methoxyethyl, or allyl. Still in other instances, X⁴ is alkoxide, Cl, or F. Typically, X⁴ is methoxide or Cl.

Another aspect of the invention provides a method for treating scleroderma or a related autoimmune or a fibrotic condition in a subject by administering a therapeutically effective amount of a compound of a formula:

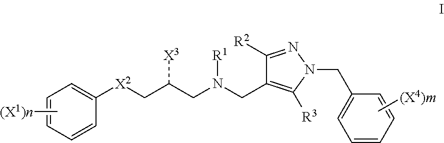

where
each of n and m is independently an integer from 0 to 5; typically each of m and n is independently an integer of 0-4; often each of m and n is independently an integer of 0-2;
each X¹ is independently alkoxide, optionally-substituted alkyl, or alkenyl;
X² is O, NR^{a}, or S;
X³ is —OR^{b}, —SR^{b}, or —NR^{b}R^{c};
each X⁴ is independently halide or alkoxide; and
each of R^{a}, R^{b}, R^{c}, R¹, R², and R³ is independently hydrogen or alkyl.

Some aspects of the invention include treating a clinical condition associated with scleroderma or related autoimmune or a fibrotic condition in a patient. Other clinical conditions that can be treated include lupus and morphea.

### BRIEF DESCRIPTION OF THE DRAWINGS

FIG. 1 is representative immunostaining images for TLR4 expression in epidermis and dermis of the SSc patients at ×40 magnification.

FIG. 2 is representative images at ×100 magnification of SSc skin biopsy showing elevated accumulation of TLR4 ligand HA in SSc dermis.

FIG. 3 is a bar graph showing TLR4-dependent stimulation of IL-8 mRNA in dermal fibroblasts.

FIG. 4 is a bar graph (left panel) and immunohistochemistry (right panel) showing TLR4 accumulation in the skin associated with bleomycin-induced scleroderma.

FIG. 5 is representative images showing TLR4 accumulation in the lungs associated with bleomycin-induced scleroderma compared to control.

FIG. 6 is hematoxylin and eosin (H&E) stained images showing effect of TLR4 inhibitor on Bleomycin-induced skin fibrosis on mice.

FIG. 7 is Mason's Trichrome stained images showing effect of TLR4 inhibitor on Bleomycin-induced skin fibrosis on mice.

FIG. 8 is a bar graph showing the effect of a compound of Formula I in normal fibroblasts stimulated with potent TLR4 ligand LPS with or without TGF-β.

FIG. 9 is a bar graph showing the effect of a compound of Formula I in three scleroderma patients.

### DETAILED DESCRIPTION OF THE INVENTION

### Definitions

“Alkyl” refers to a saturated linear monovalent hydrocarbon moiety of one to twelve, typically one to six, carbon atoms or a saturated branched monovalent hydrocarbon moiety of three to twelve, typically three to six, carbon atoms. Exemplary alkyl group include, but are not limited to, methyl, ethyl, n-propyl, 2 propyl, tert-butyl, pentyl, and the like.

“Optionally-substituted alkyl” refers to an alkyl group as defined herein in which one or more hydrogen atom is optionally replaced with a substituent such as halide, hydroxyl, alkoxy, or other heteroatom substituent.

“Alkylene” refers to a saturated linear divalent hydrocarbon moiety of one to twelve, typically one to six, carbon atoms or a saturated branched divalent hydrocarbon moiety of three to twelve, typically three to six, carbon atoms. Exemplary alkyleme group include, but are not limited to, methylene, ethylene, propylene, butylene, pentylene, and the like.

“Alkenyl” refers to a linear monovalent hydrocarbon moiety of two to ten carbon atoms or a branched monovalent hydrocarbon moiety of three to ten carbon atoms, containing at least one double bond, e.g., ethenyl, propenyl, and the like.

“Alkenyl alkyl” refers to a moiety of the formula —R^{a}-R^{b}, where R^{a} is alkylene and R^{b} is alkenyl as defined herein.

“Alkoxy” refers to a moiety of the formula —ORⁿ, where Rⁿ is alkyl as defined herein.

“Alkoxyalkyl” refers to a moiety of the formula —R^{p}—O—R^{q}, where R^{p} is alkylene and R^{q} is alkyl as defined herein.

“Antagonist” refers to a compound or a composition that attenuates the effect of an agonist. The antagonist can bind reversibly or irreversibly to a region of the receptor in common with an agonist. Antagonist can also bind at a different site on the receptor or an associated ion channel. Moreover, the term “antagonist” also includes functional antagonist or physiological antagonist. Functional antagonist refers to a compound and/or compositions that reverses the effects of an agonist rather than acting at the same receptor, i.e., functional antagonist causes a response in the tissue or animal which opposes the action of an agonist. Examples include agents which have opposing effects on an intracellular second messenger, or, in an animal, on blood pressure. A functional antagonist can sometimes produce responses which closely mimic those of the pharmacological kind.

“Aryl” refers to a monovalent mono-, bi- or tricyclic aromatic hydrocarbon moiety of 6 to 15 ring atoms.

“Optionally-substituted aryl” refers to an aryl group as defined herein in which one or more aryl ring hydrogen is replaced with a non-hydrogen substituent such as halide, alkyl, cyano, hydroxy, alkoxy, etc. When two or more substituents are present in an aryl group, each substituent is independently selected.

“Aryloxy” and “arylthio” refer to a moiety of the formula —Z—Ar¹, where Ar¹ is aryl as defined herein and Z is O and S, respectively.

“Aralkyl” refers to a moiety of the formula —R^{x}R^{y} where R^{x} is an alkylene group and R^{y} is an aryl group as defined herein. Exemplary aralkyl groups include, but are not limited to, benzyl, phenylethyl, 3-(3-chlorophenyl)-2-methylpentyl, and the like.

“Chiral center” (i.e., stereochemical center, stereocenter, or stereogenic center) refers to an asymmetrically substituted atom, e.g., a carbon atom to which four different groups are attached. The ultimate criterion of a chiral center, however, is nonsuperimposability of its mirror image.

“Cycloalkyl” refers to a non-aromatic, typically saturated, monovalent mono-, bi- or tri-cyclic hydrocarbon moiety of three to twenty ring carbons. The cycloalkyl can be optionally substituted with one or more, typically one, two, or three, substituents within the ring structure. When two or more substituents are present in a cycloalkyl group, each substituent is independently selected. Exemplary cycloalkyl groups include, but are not limited to, cyclopropyl, cyclobutyl, cyclopentyl, norbornyl, adamantyl, cyclohexyl, cyclooctyl, etc.

“(Cycloalkyl)alkyl” refers to a moiety of the formula —R^{v}R^{w} where R^{v} is an alkylene group and R^{w} is a cycloalkyl group as defined herein. Exemplary cycloalkylalkyl groups include, but are not limited to, cyclopropylmethyl, cyclohexylpropyl, 3-cyclohexyl-2-methylpropyl, and the like.

The terms “halo,” “halogen” and “halide” are used interchangeably herein and refer to fluoro, chloro, bromo, or iodo.

“Haloalkyl” refers to an alkyl group as defined herein in which one or more hydrogen atom is replaced by same or different halo atoms. The term “haloalkyl” also includes perhalogenated alkyl groups in which all alkyl hydrogen atoms are replaced by halogen atoms. Exemplary haloalkyl groups include, but are not limited to, —CH₂Cl, —CF₃, —CH₂CF₃, —CH₂CCl₃, and the like.

“Hetero-substituted alkyl” refers to an alkyl group as defined herein that contains one or more heteroatoms such as N, O, or S. Such heteroatoms can be hydroxy, alkoxy, amino, mono- or di-alkyl amino, thiol, alkylthiol, etc.

“Hydroxyalkyl” refers to an alkyl group having one or more hydroxyl substituent.

“Enantiomeric excess” refers to the difference between the amount of enantiomers. The percentage of enantiomeric excess (% ee) can be calculated by subtracting the percentage of one enantiomer from the percentage of the other enantiomer. For example, if the % ee of (R)-enantiomer is 99% and % ee of (S)-enantiomer is 1%, the % ee of (R)-isomer is 99%-1% or 98%.

“Leaving group” has the meaning conventionally associated with it in synthetic organic chemistry, i.e., an atom or a group capable of being displaced by a nucleophile and includes halo (such as chloro, bromo, and iodo), alkanesulfonyloxy, arenesulfonyloxy, alkylcarbonyloxy (e.g., acetoxy), arylcarbonyloxy, mesyloxy, tosyloxy, trifluoromethanesulfonyloxy, aryloxy (e.g., 2,4-dinitrophenoxy), methoxy, N,O-dimethylhydroxylamino, and the like.

“Pharmaceutically acceptable excipient” refers to an excipient that is useful in preparing a pharmaceutical composition that is generally safe, non-toxic and neither biologically nor otherwise undesirable, and includes excipient that is acceptable for veterinary use as well as human pharmaceutical use.

“Pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-lcarboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

The terms “pro-drug” and “prodrug” are used interchangeably herein and refer to any compound which releases an active parent drug according to Formula I in vivo when such prodrug is administered to a mammalian subject. Prodrugs of a compound of Formula I are prepared by modifying one or more functional group(s) present in the compound of Formula I in such a way that the modification(s) may be cleaved in vivo to release the parent compound. Prodrugs include compounds of Formula I wherein a hydroxy, amino, or sulfhydryl group in a compound of Formula I is bonded to any group that may be cleaved in vivo to regenerate the free hydroxyl, amino, or sulfhydryl group, respectively. Examples of prodrugs include, but are not limited to, esters (e.g., acetate, formate, and benzoate derivatives), carbamates (e.g., N,N-dimethylaminocarbonyl) of hydroxy functional groups in compounds of Formula I, and the like.

“Protecting group” refers to a moiety, except alkyl groups, that when attached to a reactive group in a molecule masks, reduces or prevents that reactivity. Examples of protecting groups can be found in T. W. Greene and P. G. M. Wuts, Protective Groups in Organic Synthesis, 3rd edition, John Wiley & Sons, New York, 1999, and Harrison and Harrison et al., Compendium of Synthetic Organic Methods, Vols. 1-8 (John Wiley and Sons, 1971-1996), which are incorporated herein by reference in their entirety. Representative hydroxy protecting groups include acyl groups, benzyl and trityl ethers, tetrahydropyranyl ethers, trialkylsilyl ethers and allyl ethers. Representative amino protecting groups include, formyl, acetyl, trifluoroacetyl, benzyl, benzyloxycarbonyl (CBZ), tert-butoxycarbonyl (Boc), trimethyl silyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC), and the like.

“Corresponding protecting group” means an appropriate protecting group corresponding to the heteroatom (i.e., N, O, P or S) to which it is attached.

“A therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

“Treating” or “treatment” of a disease includes: (1) preventing the disease, i.e., causing the clinical symptoms of the disease not to develop in a mammal that may be exposed to or predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

When describing a chemical reaction, the terms “treating”, “contacting” and “reacting” are used interchangeably herein, and refer to adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

As used herein, the terms “those defined above” and “those defined herein” when referring to a variable incorporates by reference the broad definition of the variable as well as any narrow and/or preferred, more preferred and most preferred definitions, if any.

The term “a derivative or an analog thereof” refers to those compounds that are derived from or having a similar core structure and retain all of the biological activity of the compound to which they are referred to. The term “all of the biological activity” refers to biological activities referred to herein when discussing the compound, e.g., TLR antagonistic property, etc.

### Scleroderma

Fibrosis, the hallmark of systemic sclerosis (SSc), represents the transformation of normal wound-healing into a deregulated self-sustaining process. The present inventors believe that development of effective anti-fibrotic treatments require understanding the pathological regulation of fibroblast activation and the factors that promote repair by scarring rather than healing. As discussed herein, the present inventors have elucidated molecular mechanisms that underlie normal regulation of collagen synthesis and its deregulation in SSc.

It is believed that fibroblast TLR signaling triggered by endogenous ligands underlies intractable fibrosis in SSc. In fact, it has been shown that tissue accumulation of DAMPs drives sustained fibroblast activation, transforming self-limited repair and regeneration into a vicious cycle of fibrogenesis. Normally, tissue injury triggers rapid and efficient repair. Recurrent injury leads to persistent inflammation, reactive oxygen species (ROS), increased TLR4 expression and tissue damage accompanied by generation of endogenous TLR4 ligands such as hyaluronan fragments, biglycan, Tenascin C, fibronectin-EDA and HMGB1. These danger signals trigger fibroblast TLR4 signaling, and their persistence transforms self-limited repair into refractory fibrosis.

The present inventors have discovered that fibroblasts expression can be activated through TLR4 in much the same way that immune cells recognize microbial pathogen-associated patterns. Furthermore, the present inventors have discovered that tissue expression of TLR4, and its putative endogenous ligands hyaluronic acid, Tenascin C and fibronectin-EDA, were markedly elevated in patients with SSc. Accordingly, inhibiting TLR4 (e.g., by blocking TLR4 signaling) can reduce or eliminate fibrosis, and thus can be used as an effective therapy for SSc. As disclosed herein, the present inventors have established the role of TLR4 signaling in murine scleroderma models using TLR4 inhibitors. These studies show that targeting TLR4 activity (e.g., by inhibiting TLR4 activity) redirects aberrant fibrogenesis for the treatment of SSc.

As stated above, TLR4 activation in SSc transforms self-limited repair into intractable fibrosis. Accordingly, the anti-fibrotic activity of a TLR4 inhibitor can be used for the treatment of SSc.

The present inventors have also discovered that accumulation of endogenous TLR4 ligands in injured tissue drives TLR4-mediated fibrosis amplification and persistence in SSc resulting in tissue damage and organ failure. Therefore, targeting (e.g., inhibiting) TLR4 is an effective anti-fibrotic therapy. Without being bound by any theory, a TLR4 inhibitor blocks TLR4 signaling in fibroblasts, prevents progression of fibrosis and serves as an effective therapy in scleroderma.

Some aspects of the invention provide insight into the role of TLR4 signaling in the pathogenesis of SSc, and also provide anti-fibrotic treatments for SSc.

Normal fibroblasts are activated by TGF-β, which induces the entire repertoire of profibrotic genes. Collagen regulation by TGF-β involves transcription factors Smad2/3, c-Abl, Egr-1 and Egr-2 that stimulate, and p53, BAMBI, Nab2 and PPAR-γ. Moreover, the present inventors have discovered the interplay among Smad2/3, p53, C/EBPB, Egr-1 and Egr-2, and the transcriptional coactivator p300 in the context of fibrogenesis.

Some aspects of the invention is based on the discovery by the present inventors using DNA microarrays to analyze genome-wide transcriptional profiles in SSc skin. DNA microarray analysis revealed that a subset of SSc patients showed a “TGF-β signature”. In these cases, the expression of TLR4 target genes in the skin was up-regulated in the inflammatory SSc subset, thereby indicating that TGF-β plays a significant role in the pathogenesis of fibrosis in SSc. The present inventors have delineated the TGF-β signal pathways operative during fibrogenesis. In particular, experiments showed that upon ligand-induced phosphorylation by the ALK5 transmembrane TGF-β receptor, cytosolic Smad2/3 form heteromeric complexes with Smad4 and shuttle into the nucleus where they bind to consensus SBE sequences to regulate COL1A2 transcription. Ghosh et al., J Cell Physiol., 2007, 213(3), 663-71. The present inventors have also discovered a molecular function of Smad and its dysregulation in SSc. It was also discovered that Egr-1 is also a TGF-β target with a role in collagen regulation, and implicated epigenetic modifications as important pathways. Because these pathways play a role in SSc, they can also be targeted for SSc therapy.

There are a wide variety of factors responsible for transforming self-limited tissue repair that is the hallmark of normal wound healing into a spatially and temporally self-amplifying process that is the hallmark of SSc. Exemplary factors include impaired expression, and localization and function of cell-intrinsic repressors of fibroblast activation. As disclosed herein, the present inventors have discovered an additional factor in progressive fibrosis is innate immune recognition signaling in fibroblasts via TLR4 and their endogenous ligands. Again without being bound by any theory, it is believed that by engaging fibroblast TLR4 in the injured tissue, endogenous ligands elicit profibrotic responses. In this manner, fibroblast TLR4 signaling in injured tissue causes sustained fibroblast activation and persistent, rather than self-limited, fibrogenic response, leading to intractable fibrosis.

Pattern recognition receptors (PRRs) are important sensors for exogenous danger. The family of PRRs includes lectin receptors, scavenger receptors such as CD36 and MARCO and TLRs. Recent studies showed innate immune recognition signaling in fibrosis. Activation of hepatic stellate cell TLR4 by high-dose LPS plays critical role in liver fibrosis, and sensitization of quiescent stellate cells to TGF-β has been implicated as the underlying mechanism. Moreover, the expression of both Egr-1 and Egr-2, early immediate transcription factors involved in fibrogenesis, was also induced via TLR4.

It has been shown that endogenous TLR4 ligands form three broad classes: (i) ECM-derived molecules such as hyaluronan (HA) and its small molecular weight degradation products called sHA, alternatively spliced extra domain A of fibronectin (Fn-EDA) and biglycan; (ii) stress proteins such as HMGB1; and (iii) nucleic acids and immune complexes. Each of these might be present at elevated levels at sites of tissue injury.

Some studies have shown a strong association of abnormal TLR expression and its endogenous ligand accumulation with fibrosis and SSc. Hyaluronan (HA) is a negatively charged multifunctional glycosaminoglycan composed of repeating disaccharides of glucuronic acid and glucosamine. Under physiologic conditions, HA exists as a high MW polymer (10⁶ KDa). It has been shown that injury, environmental toxins, ischemia and/or oxidative stress cause HA depolymerization via enzymatic degradation, and accumulation of low MW sHA fragments (10⁵ KDa) that can regulate cell proliferation, migration and inflammatory gene expression. Other studies have shown that sHA fragments engage TLR4 and induce MIP-1, MCP-1 and IL-8 in a variety of cell types. Levels of HA were elevated in injured kidneys, in the BAL, and in the serum in SSc.

The present inventors have found markedly elevated HA accumulation in the lesional skin from all SSc skin biopsies examined to date. Fibronectins are multifunctional ECM glycoproteins with distinct tissue and plasma forms. The spliced Fn-EDA variant has been implicated in wound healing and myofibroblast differentiation. It has been shown that Fn-EDA was elevated in pulmonary fibrosis, and in SSc lesional skin, where it co-localized with myofibroblasts. In experimental lung injury, Fn-EDA accumulation preceded fibrosis, and mice lacking the Fn-EDA domain showed defective cutaneous wound healing and protection from bleomycin-induced lung fibrosis. TLR4 null mice were also protected from cardiac and renal fibrosis. HMGB 1 levels were elevated in fibrotic lungs and in the lesional skin of patients with SSc. Biglycan has been shown to induce TLR4 activation. Together these observations indicate a role for endogenous TLR4 signaling in the pathogenesis of fibrosis in SSc.

The present inventors have discovered that accumulation of endogenous TLR4 ligands in injured tissue leads to TLR4-mediated fibrosis amplification and persistence in SSc, resulting in tissue damage and organ failure. Accordingly, some aspects of the invention provide methods for treating scleroderma by administering a TLR4 inhibitor to a subject who is suffering from scleroderma.

While the scope of present invention is not limited to any particular chemical compound or composition, the following provides some known representative TLR4 inhibitors that are useful in treating scleroderma. In one particular embodiment of the invention, a TLR4 inhibitor is a compound of the formula:

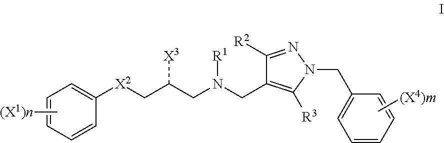

where
each of n and m is independently an integer from 0 to 5; typically each of m and n is independently an integer of 0-4; often each of m and n is independently an integer of 0-2;
each X¹ is independently alkoxide, optionally-substituted alkyl, or alkenyl;
X² is O, NR^{a}, or S;
X³ is —OR^{b}, —SR^{b}, or —NR^{b}R^{c};
each X⁴ is independently halide or alkoxide; and
each of R^{a}, R^{b}, R^{c}, R¹, R², and R³ is independently hydrogen or alkyl.

Within these embodiments, in some instances X² is O. Still in other instances, X³ is —OH. Yet in other instances, R¹, R² and R³ are alkyl. Typically, R¹, R², and R³ are methyl. Yet in other instances, X¹ is alkoxide, hetero-substituted alkyl or alkenyl-alkyl. Often X¹ is methoxide, methoxyethyl, or allyl. Still in other instances, X⁴ is alkoxide, Cl, or F. Typically, X⁴ is methoxide or Cl.

It should be appreciated that the scope of the invention is not limited to those particular embodiments disclosed above. In fact, combinations of particular embodiments described herein form other embodiments. Thus, for example, in one particularly embodiment X² is O, X³ is —OH; R¹, R² and R³ are methyl; X¹ is methoxide; and X⁴ is methoxide. In this manner, a variety of compounds are embodied within the present invention.

Synthesis of various TLR4 inhibitors and methods for determining whether a particular compound is a TLR4 inhibitor is well known to one skilled in the art. See, for example, PCT Patent Application No. PCT/US10/50050, filed Sep. 23, 2010, which is incorporated herein by reference in its entirety. Thus, one skilled in the art having read the present disclosure can readily use other TLR4 inhibitors to treat scleroderma. And the scope of the invention includes such TLR4 inhibitors.

Other TLR4 compounds can be readily synthesized by one skilled in the art having read the present disclosure, for example, by changing one or more substituents on the compounds. Synthesis of a TLR4 modulating compound can include changing one or more substituents that are present in the starting compounds, by adding an appropriate substituent on one of the intermediates or by adding the substituent after formation of the final products by known methods of substitution or conversion reactions. For example, nitro groups can be added by nitration and the nitro group can be converted to other groups, such as amino by reduction, and halogen by diazotization of the amino group and replacement of the diazo group with halogen or simply by halogenation reaction. Acyl groups can be added by Friedel-Crafts acylation. The acyl groups can then be transformed to the corresponding alkyl groups by various methods, including the Wolff-Kishner reduction and Clemmenson reduction. Amino groups can be alkylated to form mono- and di-alkylamino groups; and mercapto and hydroxy groups can be alkylated to form corresponding ethers. Primary alcohols can be oxidized by oxidizing agents known in the art to form carboxylic acids or aldehydes, and secondary alcohols can be oxidized to form ketones. Thus, substitution or alteration reactions can be employed to provide a variety of substituents throughout the molecule of the starting material, intermediates, or the final product, including isolated products.

Additionally, as will be apparent to those skilled in the art, conventional protecting groups may be necessary to prevent certain functional groups from undergoing undesired reactions. The choice of a suitable protecting group for a particular functional group, as well as suitable conditions for protection and deprotection, are well known in the art. For example, numerous protecting groups, and their introduction and removal, are described in T. W. Greene and G. M. Wuts, Protecting Groups in Organic Synthesis, 3rd ed., John Wiley & Sons, New York, 1999, and references cited therein, all of which are incorporated herein by reference in their entirety.

Since TLR4 inhibitors can have certain substituents which are necessarily present, the introduction of each substituent is, of course, dependent on the specific substituents involved and the chemistry necessary for their formation. Thus, consideration of how one substituent would be affected by a chemical reaction when forming a second substituent would involve techniques familiar to one of ordinary skill in the art. This would further be dependent on the ring involved.

In some instances, a racemic mixture of TLR4 inhibitors can be prepared and the desired (+)- or (−)-isomer can be resolved or separated (i.e., enantiomerically enriched) using any of the variety of chiral resolution methods known to one skilled in the art. Such resolution methods are described, for example, in the four volume compendium Optical Resolution Procedures for Chemical Compounds: Optical Resolution Information Center, Manhattan College, Riverdale, N.Y., and in Enantiomers, Racemates and Resolutions, Jean Jacques, Andre Collet and Samuel H. Wilen; John Wiley & Sons, Inc., New York, 1981, which are incorporated herein in their entirety.

In some resolution methods, a racemic mixture is converted to a mixture of diasteromers by attachment, either chemically or enzymatically, of a relatively enantiomerically pure moiety. Unlike enantiomers, most diastereomers have different physical properties, e.g., solubility, boiling point, affinity (e.g., to chromatography columns and enzymes), and the like. These different physical properties can be used to separate one diastereoisomer from another, for example, by fractional crystallization, distillation, chromatography, kinetic resolution using an enzyme, and the like.

Alternatively, a TLR4 inhibitor can be synthesized enantioselectively starting from enantiomerically pure or enriched starting material. In some embodiments, the enantiomeric excess of a TLR4 inhibitor is at least 90%, typically at least 95%, and often at least 98%.

When a TLR4 inhibitor contains an olefin moiety and such olefin moiety can be either cis- or trans-configuration, the TLR4 inhibitor can be synthesized to produce cis- or trans-olefin, selectively, as the predominant product. Alternatively, the TLR4 inhibitor containing an olefin moiety can be produced as a mixture of cis- and trans-olefins and separated using known procedures, for example, by chromatography as described in W. K. Chan, et al., J. Am. Chem. Soc., 1974, 96, 3642, which is incorporated herein in its entirety.

The TLR4 inhibitors form salts with acids when a basic amino function is present and salts with bases when an acid function, e.g., carboxylic acid or phosphonic acid, is present. All such salts are useful in the isolation and/or purification of the new products. Of particular value are the pharmaceutically acceptable salts with both acids and bases. Suitable acids include, for example, hydrochloric, oxalic, sulfuric, nitric, benzenesulfonic, toluenesulfonic, acetic, maleic, tartaric and the like which are pharmaceutically acceptable. Basic salts for pharmaceutical use include Na, K, Ca and Mg salts.

Methods for producing various TLR4 inhibitors are readily available from various journal articles and patents, which can be readily obtained by, for example, searching chemical abstract services data base, e.g., CAS online.

The TLR4 inhibitors can be administered to a patient to achieve a desired physiological effect. Typically the patient is a mammal, often human. The TLR4 inhibitor can be administered in a variety of forms adapted to the chosen route of administration, i.e., orally or parenterally. Parenteral administration in this respect includes administration by the following routes: intravenous; intramuscular; subcutaneous; intraocular; intrasynovial; transepithelially including transdermal, ophthalmic, sublingual and buccal; topically including ophthalmic, dermal, ocular, rectal, and inhalation (e.g., via insufflation and aerosol); intraperitoneal; rectal systemic, and central (e.g., intrathecal, such as into the cerebrospinal fluid around the spinal cord, and intracerebral into brain or C SF of the brain).

The TLR4 inhibitor can be orally administered, for example, with an inert diluent or with an assimilable edible carrier, or it can be enclosed in hard or soft shell gelatin capsules, or it can be compressed into tablets, or it can be incorporated directly with the food of the diet. For oral therapeutic administration, the TLR4 inhibitor may be incorporated with excipient and used in the form of ingestible tablets, buccal tablets, troches, capsules, elixirs, suspensions, syrups, wafers, and the like. Such compositions and preparation can contain at least 0.1% of TLR4 inhibitor. The percentage of the compositions and preparation can, of course, be varied and can conveniently be between about 1 to about 10% of the weight of the unit. The amount of TLR4 inhibitor in such therapeutically useful compositions is such that a suitable dosage will be obtained. Typical compositions or preparations are prepared such that an oral dosage unit form contains from about 1 to about 1000 mg of TLR4 inhibitor.

The tablets, troches, pills, capsules and the like can also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin can be added or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the TLR4 inhibitor can be incorporated into sustained-release preparations and formulation.

The TLR4 inhibitor can also be administered parenterally. Solutions of the TLR4 inhibitor as a free base or pharmacologically acceptable salt can be prepared in water suitably mixed with a surfactant such as hydroxypropylcellulose. Dispersion can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It can be stable under the conditions of manufacture and storage and must be preserved against the contaminating action of microorganisms such as bacterial and fungi. The carrier can be a solvent of dispersion medium containing, for example, water, ethanol, polyol (e.g., glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, e.g., sugars or sodium chloride. Prolonged absorption of the injectable compositions of agents delaying absorption, e.g., aluminum monostearate and gelatin.

Sterile injectable solutions are prepared by incorporating the TLR4 inhibitor in the required amount in the appropriate solvent with various other ingredients enumerated above, as required, followed by filtered sterilization. Generally, dispersions are prepared by incorporating the various sterilized TLR4 inhibitor into a sterile vehicle which contains the basic dispersion medium and the required other ingredients from those enumerated above. In the case of sterile powders for the preparation of sterile injectable solutions, typical methods of preparation are vacuum drying and the freeze drying technique which yield a powder of the TLR4 inhibitor plus any additional desired ingredient from previously sterile-filtered solution thereof.

The TLR4 inhibitors can be administered to a mammal alone or in combination with pharmaceutically acceptable carriers, as noted above, the proportion of which is determined by the solubility and chemical nature of the TLR4 inhibitor, chosen route of administration and standard pharmaceutical practice.

The physician will determine the dosage of the TLR4 inhibitors which will be most suitable for prophylaxis or treatment and it will vary with the form of administration and the particular TLR4 inhibitor chosen, and also, it will vary with the particular patient under treatment. The physician will generally wish to initiate treatment with small dosages by small increments until the optimum effect under the circumstances is reached. The therapeutic dosage can generally be from about 0.1 to about 1000 mg/day, and typically from about 10 to about 100 mg/day, or from about 0.1 to about 50 mg/Kg of body weight per day and often from about 0.1 to about 20 mg/Kg of body weight per day and can be administered in several different dosage units. Higher dosages, on the order of about 2× to about 4×, may be required for oral administration.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

Evidence for TLR Signaling in SSc Skin:

To examine global gene expression in SSc skin biopsies, DNA microarray analysis was performed. RNA was isolated from lesional and non-lesional skin biopsies from 34 individuals using RNAlater (Ambion) and following cRNA synthesis and labeling, hybridized to 44,000 element Agilent DNA oligonucleotide microarrays. Unsupervised hierarchical cluster analysis was then performed. (Data not shown). The analysis of data revealed there was significant diversity of gene expression among individual SSc patients. Data were analyzed by focusing on the expression of genes that were identified previously as TLR4-inducible target genes in mesenchymal cells. There was significant up-regulation of TLR4 target genes such as inflammatory cytokines/chemokines and their receptors, TLRs and endogenous TLR ligands such as HMGB1, and the early immediate transcription factors Egr1 and Egr-2. These TLR4-inducible target genes were most up-regulated in the inflammatory SSc subset, indicating activated TLR4 signaling in the skin.

Elevated TLR4 Expression in SSc Skin:

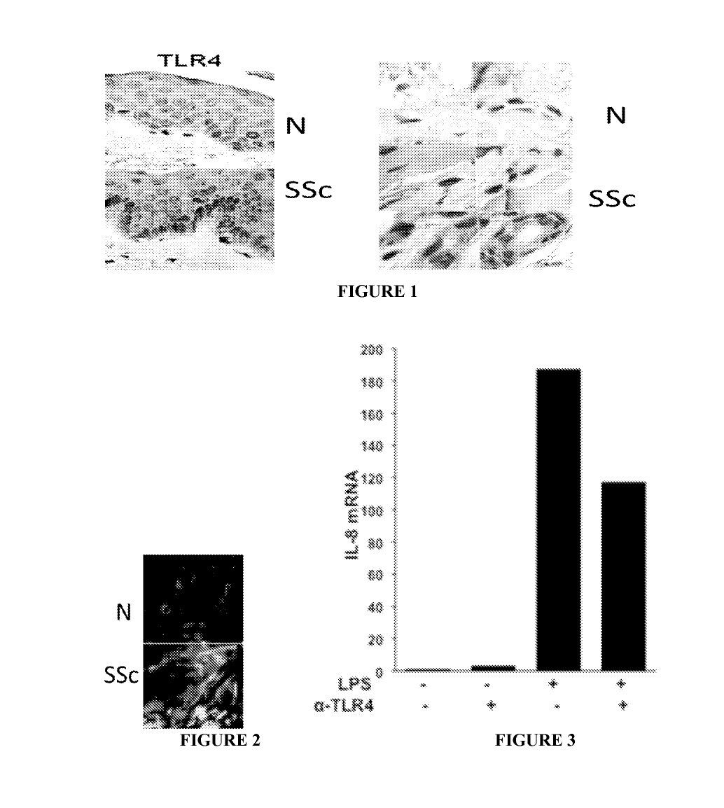

Immunohistochemistry was used to characterize the tissue expression of TLRs and their putative endogenous ligands in SSc. Skin biopsies were examined from five healthy adults and from five patients with SSc. When control and SSc skin biopsies were compared, it was found that epidermal expression of TLR4 was significantly increased in all SSc skin biopsies (data not shown, but see FIG. 1 which shows increased immunostaining for TLR4 in SSc epidermis and dermis). In contrast to normal dermis (N in FIG. 1) where little TLR4 expression was seen, increased dermal cell TLR4 expression was evident in biopsies from SSc patients (“SSc”). The increased level of cutaneous TLR4 in SSc is believed to reflect autocrine and paracrine stimulation of TLR4 gene expression by locally acting inflammatory cytokines, TGF-β or hypoxia in the fibrotic milieu.

Elevated Accumulation of TLR4 Ligand HA in SSc Dermis:

Immunofluorescence was used to characterize the tissue expression of TLR4 endogenous ligand HA in SSc. Skin biopsies were examined from five healthy adults and from five patients with SSc by immunofluorescence. When control and SSc skin biopsies were compared, it was found that HA expression was significantly increased in all SSc skin biopsies (see FIG. 2).

Dermal Fibroblasts Respond to TLR4 Ligand:

Microbial TLR4 ligands were used to investigate TLR activation in fibroblasts. Confluent normal dermal fibroblasts at low passage were incubated in endotoxin-free media with the microbial TLR ligands peptidoglycan from S. aureus or ultrapure LPS 10 ng/ml (Sigma). Following 4 h incubation, the cultures were harvested and total RNA was isolated and analyzed by real-time PCR. The results showed that LPS, but not PGN, induced a dramatic up-regulation of IL-8 mRNA (see FIG. 3). Preincubation of the cultures with low concentration of an antibody to TLR4 (Santa Cruz, 5 μg/ml) significantly suppressed the stimulatory effect.

Elevated TLR Expression in Bleomycin-Induced Scleroderma:

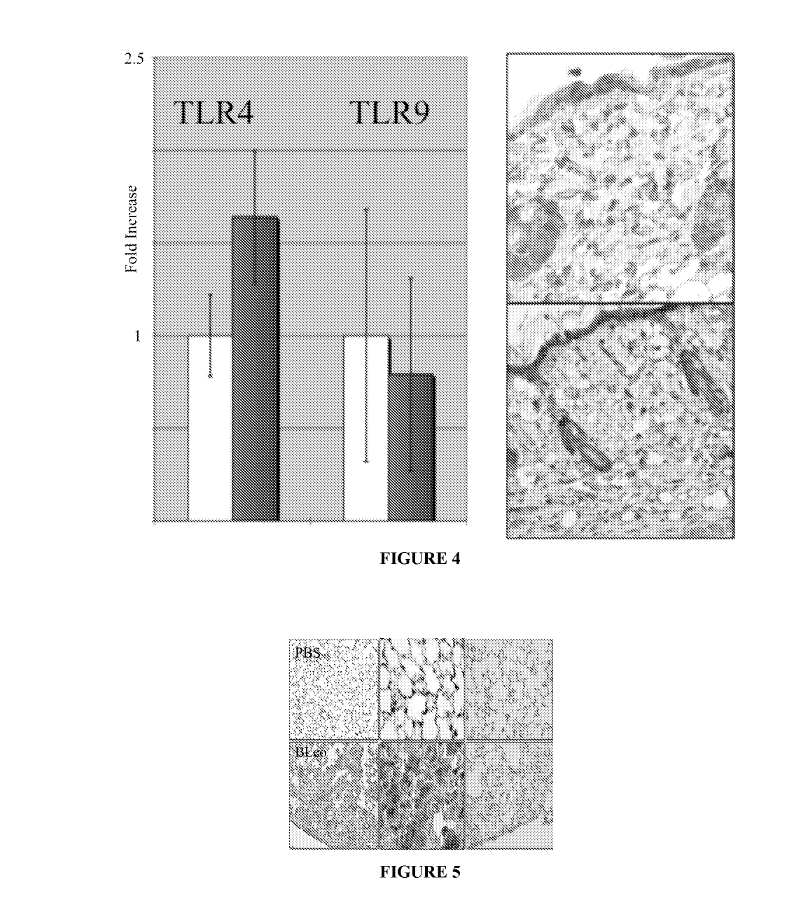

TLR expression in a mouse model of scleroderma was examined. Daily subcutaneous injections of bleomycin resulted in the development of sclerderma-like skin induration and focal lung fibrosis in 6-12 week old C57/BL mice. From the skin tissue, RNA was isolated and was subjected to qPCR. Immunohistochemistry was used to characterize TLR expression in this model. Tissue was formalin-fixed, embedded in paraffin and sectioned. The optimal concentration for each anti-TLR antibody was determined in pilot experiments. qPCR results from mouse skin tissue showed that TLRs 4 and 9 were detectable in mouse skin, however only TLR4 expression was evident in bleomycin injected lesional skin (see FIG. 4. Note positive immunostaining of fibroblastic cells in the dermis, in bleomycin-treated mice. White bars and red bars show qPCR from PBS and bleomcyin-treated mice, respectively).

Elevated TLR4 Expression in the Lungs in Bleomycin-Induced Scleroderma:

6-12 week old C57 mice were injected with PBS (FIG. 5, upper panels) or bleomycin s.c (FIG. 5, lower panels) for 28 days, followed by 14 days recovery. Lungs were then harvested, and processed for immunohistochemistry using antibodies to TLR4 using antibodies to TLR4. Note strong TLR4 immunostaining of fibroblastic and monocytic cells within fibrotic lesions in the lungs from bleomycin-treated mice. FIG. 5 shows ×20 (left), 40 (right) and 80 (middle) magnification. Right panels in FIG. 5 show negative control (no primary antibody).

Effect of TLR4 Inhibitor on Bleomycin-Induced Skin Fibrosis:

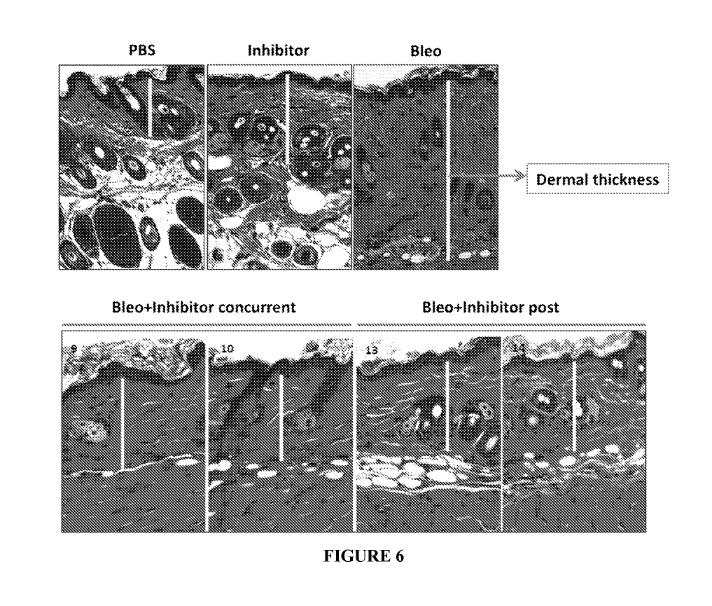

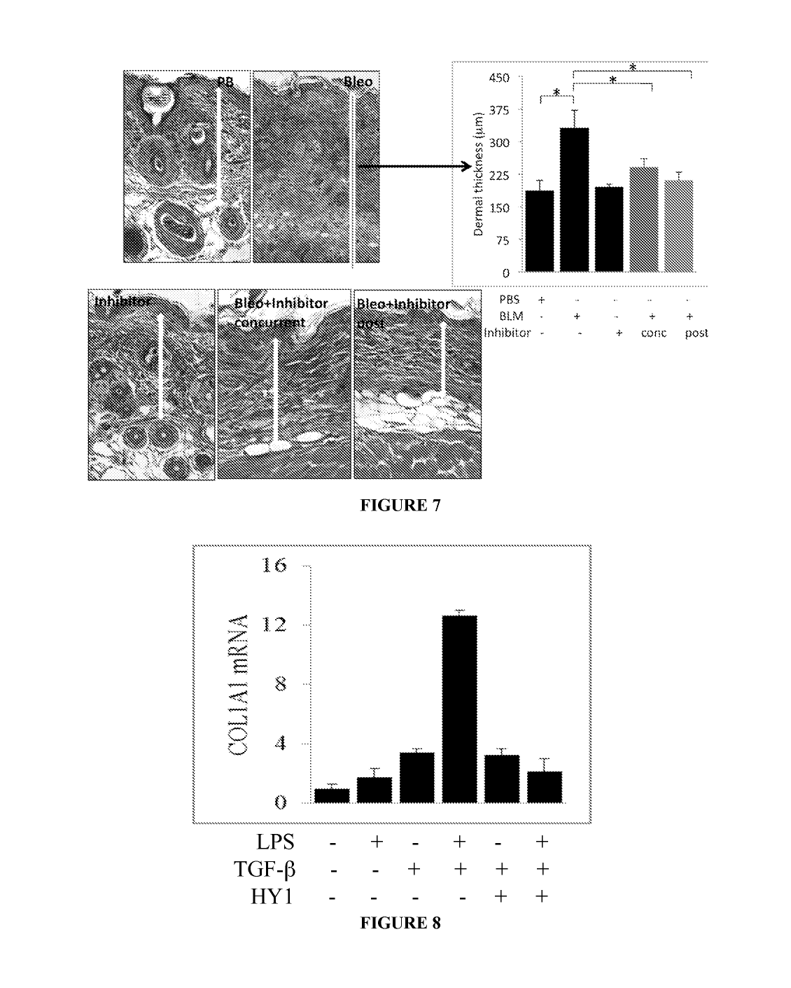

To investigate the effects of pharmacological TLR4 blockade, eight-week-old female C57Bl/6J mice (Jackson Laboratories, Sacramento, Calif.) were given daily s.c. injections of bleomycin (10 mg/kg) or PBS for 10 days, with some mice also receiving the TLR4 inhibitor Compound 1 (daily 1 mg/kg/mouse intraperitoneally) concurrently with PBS or bleomycin. Another group of mice received Compound 1 (1 mg/kg/mouse) by daily injections initiated after completion of bleomycin injections (day 10) to examine the effect of Compound 1 on regression of established fibrosis. All the mice were sacrificed on day 28 and skin was harvested for analysis. Each experimental group consisted of five mice. Four mm thick sections of paraffin-embedded tissues were stained with hematoxylin and eosin (FIG. 6) or Masson's Trichrome stain (FIG. 7) for visualizing collagen. The dermal thickness (distance from the dermal-epidermal junction to the adipose layer) was determined (bar graph on FIG. 7), and results shown as the means±s.d. of triplicate determinations/hpf from five mice/group are considered as statistically significant (p<0.01).

There was no weight loss or other evidence of toxicity in mice treated with Compound 1. Examination of the lesional skin from mice treated with bleomycin showed a substantial increase (˜2-fold) in dermal thickness (p<0.01), there was accumulation of densely packed collagen bundles accompanied by loss of subcutaneous adipose, and striking necrosis of the subjacent skeletal muscle (FIGS. 6 and 7, left panel). Mice treated with Compound 1 showed a significant attenuation of dermal fibrosis (p<0.01) (FIGS. 6 and 7, right panel). Similar results were seen with concurrent and post-bleomycin treated conditions.

### Results

The present inventors have discovered that fibroblast TLR4 were activated by damage-associated endogenous ligands generated during tissue damage in SSc. Accordingly, it was discovered that fibrosis can be treated by blocking TLR4 signaling. In particular, scleroderma (e.g., SSc) can be treated, for example, by using a TLR4 inhibitor.

It was discovered that in SSc, recurrent injury causes tissue damage that is accompanied by the generation and persistence of ECM breakdown products and release of cellular components. Recognition of these as danger signals by fibroblast, TLRs, in particular TLR4, is believed to be responsible for on-going fibroblast activation and progressive fibrosis rather than normal healing and regeneration. In some embodiments of the invention, TLR4, a membrane-spanning receptor protein that functions in complex with its accessory protein MD-2, is a target for therapeutic treatment. A wide variety of TLR4 inhibitors are known and are currently developed including by enantioselective synthesis using a Mannich type reaction to functionalize a pyrazole ring. In silico and cellular assay results demonstrated that a compound of Formula I, e.g., (R)-isomer of compound 2126 or HY1) selectively block TLR4 activation in live cells. It is shown that blocking TLR4 modulates (e.g., reduces or prevents) persistence and progression of fibrosis in scleroderma.

Mouse Models of Scleroderma:

There are currently no animal model that reproduce all key features (vasculopathy, autoimmunity and fibrosis) of SSc. However, particular mouse models have been extremely valuable for fibrosis studies. Bleomycin-induced scleroderma is widely used as a model for evaluating inflammatory cell and fibroblast interaction, and delineating molecular pathways. The sequence of histological changes in the skin closely recapitulates those characteristics of SSc. Furthermore, subcutaneous (s.c.) bleomycin-treated mice also develop progressive pulmonary fibrosis. The pulmonary pathology induced by s.c bleomycin is quite distinct from that induced by the commonly used intratracheal approach, which causes a fleeting bronchiolocentric lesion, rather than the progressive peripheral lesions induced by s.c bleomycin that closely resemble SSc-associated non-specific interstitial pneumonitis (NSIP).

Scleroderma was induced in healthy 8-12 week old C57Bl or DBA mice using daily s.c. bleomycin injections, following standard approaches. Mice were given a TLR4 inhibitor either at the beginning of the injections (prevention protocol), or about 1 week after initiation of the injections, when inflammation was already maximal (reversal or therapeutic protocol). At the end of the experiments, skin and lungs were harvested and processed for routine histology (H&E, trichrome and picrosirius red stains), immunohistology (paraffin-embedded or frozen samples), in situ hybridization, SIRCOL assays, and RNA isolation for real-time qPCR. Each of these experimental methodologies were optimized and were in routine use by the present inventors. At each time point, dermal inflammation was quantified and characterized. In addition, dermal thickening was compared. Collagen accumulation (Trichrome) and collagen cross-linking (Sirius red) in lesional skin and lungs were also examined, characterized and/or quantified. Changes in TLR4 expression on myofibroblasts and on inflammatory cells infiltrating lesional tissue were also examined by double immunofluorescence staining, and the accumulation of the TLR4 ligands HA (using biotinylated HABP), HMGB1, biglycan and Fn-EDA in lesional tissue were assessed. In addition, non-crosslinked fibrillar collagen content (SIRCOL assays), and collagen mRNA levels (qPCR) in lesional skin and lungs were measured.

Experiments by the present inventors using a TLR4 inhibitor showed TLR4 has a role in the development of persistent fibrosis. The present inventors have discovered that TLR4 is up-regulated in the skin in SSc patients and in mice with bleomycin-induced scleroderma, along with marked accumulation of endogenous TLR4 ligands in the lesional tissue. Based on this discovery, experiments using TLR4 inhibitor-treated mice demonstrated attenuation of skin and lung fibrosis compared to wildtype controls, and regression or resolution of fibrotic changes following the cessation of daily bleomycin injections compared to persistent or progressive fibrosis in TLR4 wildtype mice.

Effect of TLR4 Inhibitor HY1 in Scleroderma Patients Fibroblasts:

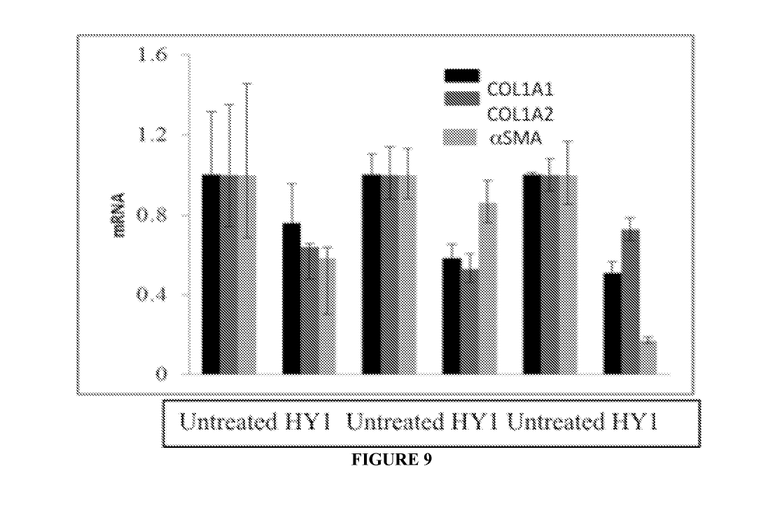

Normal skin fibroblasts were treated with a compound of Formula I in the absence or the presence of LPS (potent TLR4 ligand) and TGF-β. FIG. 8 shows effect of HY1 in normal fibroblasts stimulated with potent TLR4 ligand LPS with or without TGF-β. Compound HY1 was used in fibroblasts of three scleroderma patients. mRNA was isolated and subjected to qPCR.COL1A1, a profibrotic gene was tested for the inhibitor effect. As shown in FIG. 9, about 50% reduction in profibrotic markers was observed with HY1.

### Chemical Synthesis

All reactions were run in oven-dried or flame-dried glassware under a dry nitrogen or argon atmosphere. Methanol was distilled by simple distillation and stored over 4 Å molecular sieves. Acetone was distilled before use. Methylamine.HCl salt was dried under high-vac overnight using P₂O₅ as a decadent. All other reagents and solvents were used as received from the supplier. Flash chromatography was performed using 32-64 μm silica gel. ¹H NMR spectra were recorded at 300 MHz, 400 MHz, or 500 MHz in CDCl₃ using residual CHCl₃ (7.26 ppm) as the internal standard. ¹³C NMR spectra were recorded at 75 MHz in CDCl₃ using residual CHCl₃ (77.23 ppm) as an internal reference. Exact mass was determined using electrospray ionization.

Some of the compounds of the invention were synthesized as shown in Scheme 1. Synthesis of Compound A-1 was achieved by alkylation of the pyrazole (1-3), followed by a Mannich-like reaction to produce the tetrasubstituted pyrazole derivative (1-5). Finally, epoxide opening of compound 1-2 with the amino functionality of compound 1-5 provided Compound A-1.

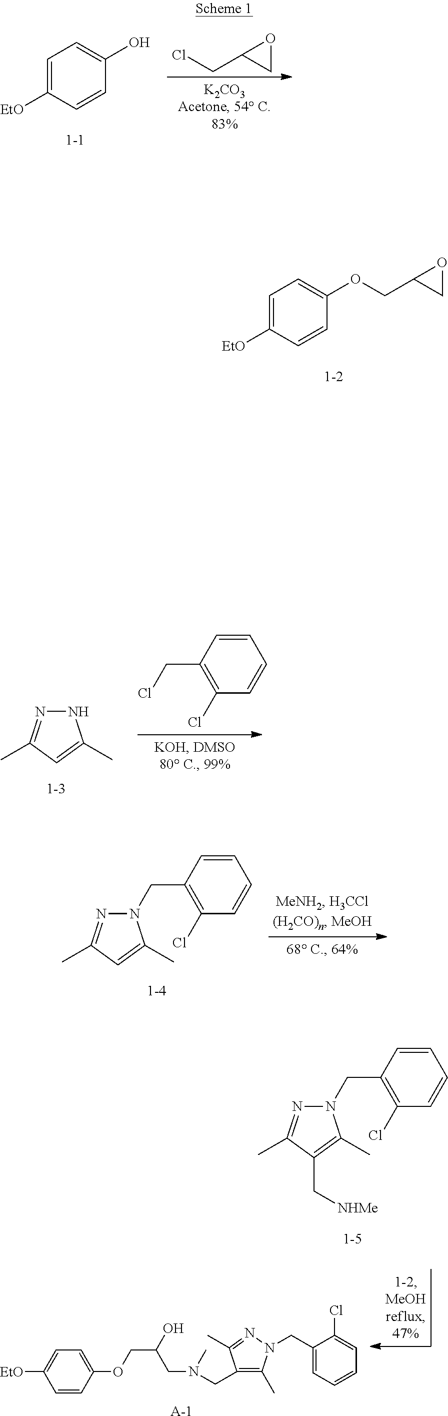

These compounds were evaluated for inhibition of the TLR4 signaling pathway in vitro and in vivo. Compound A-1 substantially inhibited TLR4 binding.

### 2-((4-ethoxyphenoxy)methyl)oxirane

4-Ethoxyphenol (0.25 g, 1.81 mmol), anhydrous potassium carbonate (0.50 g, 3.62 mmol) and epichlorohydrin (0.57 ml, 7.24 mmol) were added to acetone (4.52 ml) and the resulting heterogeneous solution was refluxed for 16 hrs. The mixture was cooled to room temperature, filtered through a pad of celite and the filtrate was concentrated under reduced pressure. The resulting oil was dissolved in toluene (20 mL), washed sequentially with water (15 mL), 5% aqueous NaOH (20 mL) and water again (20 mL). The organic layer was dried with MgSO₄ and concentrated under reduced pressure to yield 0.292 g (83%) of 2-((4-ethoxyphenoxy)methyl)oxirane as a white solid (mp=41° C.). ¹H NMR (400 MHz, CDCl₃) δ 6.91-6.77 (m, 4H), 4.17 (dd, J=11.0, 3.2, 1H), 3.98 (q, J=7.0, 2H), 3.91 (dd, J=11.0, 5.6, 1H), 3.34 (m, 1H), 2.90 (dd, J=4.9, 4.1, 1H), 2.75 (dd, J=5.0, 2.7, 1H), 1.39 (t, J=6.98, 3H). ¹³C NMR (75 MHz, CDCl₃) δ 153.72, 152.78, 115.90, 115.90, 115.59, 115.59, 69.71, 64.18, 50.49, 44.98, 15.15. HRMS (m/z): [MNa]⁺ calc. for C₁₁H₁₄O₃Na⁺, 217.08. found 217.0826.

### 1-(2-chlorobenzyl)-3,5-dimethyl-1H-pyrazole

Powdered potassium hydroxide (1.751 g, 31.2 mmol) was added to a solution of 3,5-dimethylpyrazole (2 g, 20.81 mmol) in anhydrous DMSO (10.40 ml) and the resulting heterogeneous solution was stirred for 1.5 hr at 80° C. before being cooled to room temperature. 2-Chloro benzylchloride (2.64 ml, 20.81 mmol) was then added in 6 M DMSO over 15 min, and the solution was stirred for a further 1.5 hrs. Upon completion as observed by TLC, the reaction was poured over water and the resulting aqueous phase was extracted with two 20 mL portions of CHCl₃. The combined organic layers were washed with 100 mL of water, dried with anhydrous MgSO₄ and concentrated under reduced pressure to yield 4.55 g (99%) of 1-(2-chlorobenzyl)-3,5-dimethyl-1H-pyrazole as a clear liquid. ¹H NMR (300 MHz, CDCl₃) δ 7.41-7.31 (m, 1H), 7.24-7.09 (m, 2H), 6.59-6.50 (m, 1H), 5.90 (s, 1H), 5.31 (s, 2H), 2.26 (s, 3H), 2.15 (s, 3H). ¹³C NMR (75 MHz, CDCl₃) δ 148.32, 139.96, 135.46, 131.96, 129.42, 128.76, 127.72, 127.48, 105.84, 50.12, 13.80, 11.15. HRMS (m/z): [MNa]⁺ calc for C₁₂H₁₃ClN₂Na⁺ 243.07. found 243.0651.

### 1-(1-(2-chlorobenzyl)-3,5-dimethyl-1H-pyrazol-4-yl)-N-methylmethanamine

A solution of 1-(2-chlorobenzyl)-3,5-dimethyl-1H-pyrazole (1.00 g, 4.53 mmol), paraformaldehyde (0.82 g, 27.20 mmol) and methylamine.HCl (0.92 g, 13.59 mmol) dissolved in methanol (9.06 ml) was stirred at 60° C. for 24 hrs. The mixture was cooled to room temperature and quenched with aqueous NaHCO₃ (15 mL). The aqueous layer was extracted 3 times with ether (15 mL) and the combined organic layers washed with brine (30 mL). The organic layer was dried with MgSO₄ and concentrated under reduced pressure. The resulting yellow oil was purified using flash column chromatography with 1:4:0.01 ethyl acetate:hexanes:triethylamine as eluting solvent yielding 0.73 g (62%) of 1-(1-(2-chlorobenzyl)-3,5-dimethyl-1H-pyrazol-4-yl)-N-methylmethanamine as a clear oil. ¹H NMR (300 MHz, CDCl₃) δ 7.40-7.31 (m, 1H), 7.23-7.08 (m, 2H), 6.54-6.43 (m, 1H), 5.32 (s, 2H), 3.31 (s, 2H), 2.91 (s, 1H), 2.25 (s, 3H), 2.16 (s, 3H), 2.12 (s, 3H). ¹³C NMR (75 MHz, CDCl₃) δ 148.12, 138.48, 135.57, 131.94, 129.42, 128.72, 127.61, 127.41, 114.38, 50.24, 49.08, 40.68, 12.36, 9.75. HRMS (m/z): [MH]⁺ calc for C₁₄H₁₈ClN₃, 264.13. found 264.1253.

### 1-(((1-(2-chlorobenzyl)-3,5-dimethyl-1H-pyrazol-4-yl)methyl)(methyl)amino)-3-(4-ethoxyphenoxy)propan-2-ol

2-((4-Ethoxyphenoxy)methyl)oxirane (0.06 g, 0.32 mmol) and 1-(1-(2-chlorobenzyl)-3,5-dimethyl-1H-pyrazol-4-yl)-N-methylmethanamine (0.10 g, 0.38 mmol) were dissolved in methanol (0.32 ml), warmed to 68° C. and stirred until the oxirane was consumed as observed by TLC. The solution was cooled to room temperature and the solvent removed under reduced pressure. The resulting oil was purified using flash column chromatography with 1:2:0.01 ethyl acetate:hexanes:triethylamine as the eluting solvent to yield 0.09 g (63%) of 1-(((1-(2-chlorobenzyl)-3,5-dimethyl-1H-pyrazol-4-yl)methyl)(methyl)amino)-3-(4-ethoxyphenoxy)propan-2-ol as a clear liquid. ¹H NMR (500 MHz, CDCl₃) δ 7.35 (dd, J=7.8, 1.2, 1H), 7.17 (td, J=7.7, 1.3, 1H), 7.12 (td, J=7.5, 1.2, 1H), 6.85-6.79 (m, 4H), 6.48 (dd, J=7.6, 0.9, 1H), 5.28 (s, 2H), 4.12-4.04 (m, 1H), 3.97 (q, J=7.0, 2H), 3.90 (d, J=4.9, 2H), 3.47 (d, J=13.2, 1H), 3.34-3.29 (m, 1H), 2.60 (dd, J=12.2, 9.7, 1H), 2.48 (dq, J=12.2, 3.9, 1H), 2.26 (s, 3H), 2.24 (s, 3H), 2.11 (s, 3H), 1.38 (t, J=9.1, 3H). ¹³C NMR (75 MHz, CDCl₃) δ 153.47, 153.04, 148.04, 138.67, 135.31, 131.92, 129.43, 128.79, 127.58, 127.50, 115.63, 115.63, 115.53, 115.53, 113.71, 71.25, 66.37, 64.14, 59.51, 51.70, 50.28, 42.09, 15.13, 12.36, 9.82. HRMS (m/z): [MNa]⁺ calc for C₂₅H₃₂ClN₃O₃Na⁺, 480.20. found 480.2030.

Other Compounds:

Some of the representative compound of Formula I that were prepared are listed below (some of the salts and enantiomerically enriched isomers were also prepared but are not shown separately):

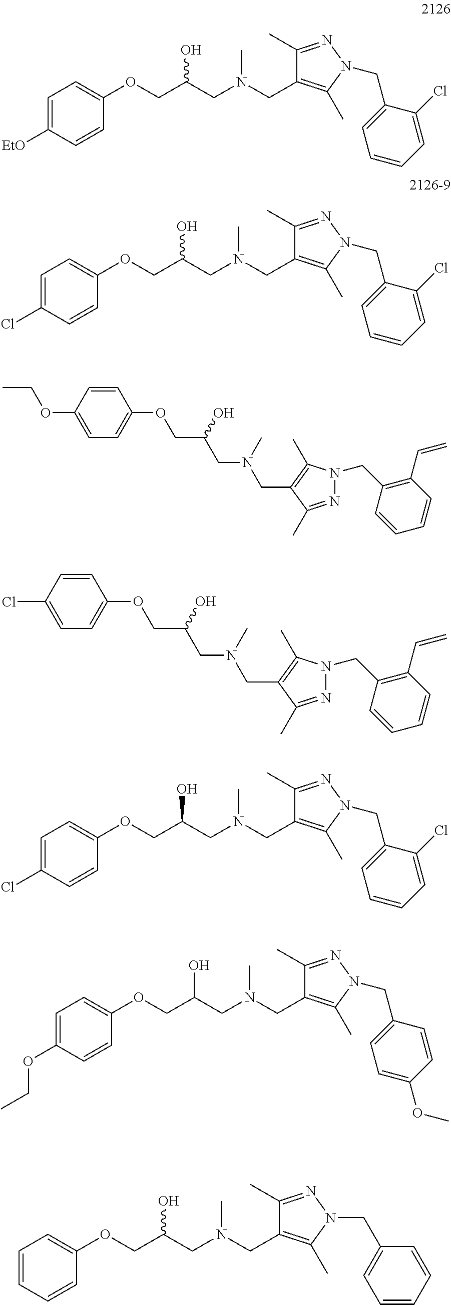

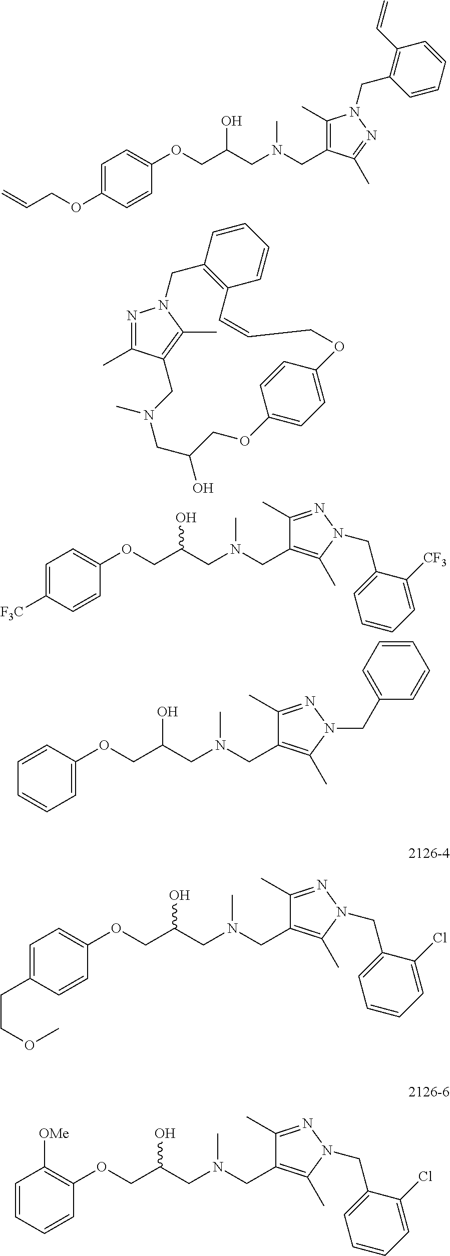

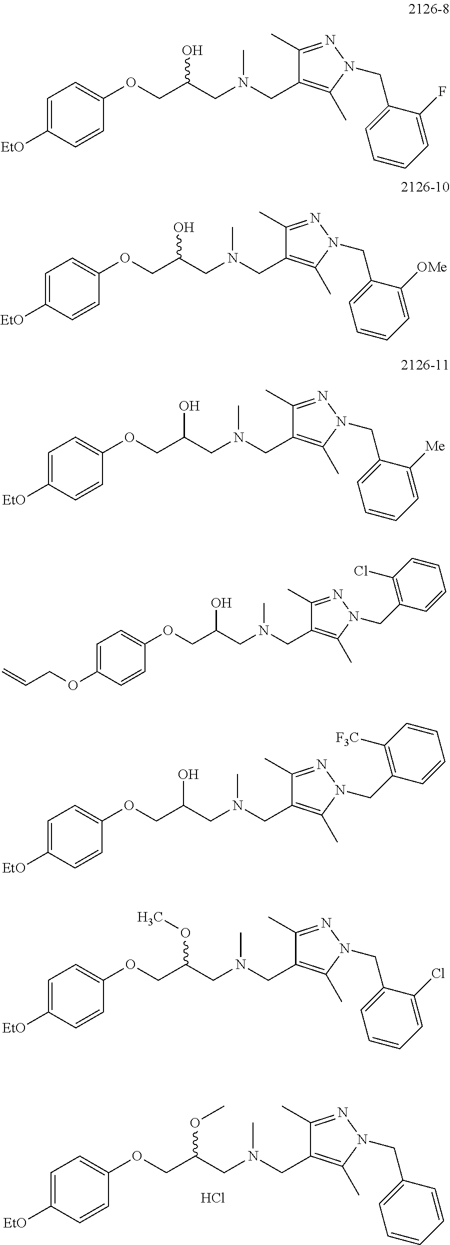

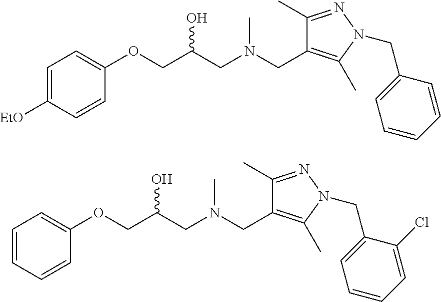 [SMILES: CCOc1ccc(OCC(O)CN(C)Cc2c(C)nn(Cc3ccccc3)c2C)cc1 ; Cc1nn(Cc2ccccc2Cl)c(C)c1CN(C)CC(O)COc1ccccc1]

Other exemplary compound of Formula I include the following:

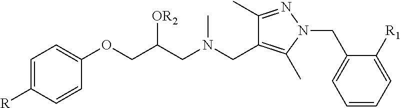

| R | R1 | R2 |
|---|---|---|
| —OH | —Cl | —H |
| —H | —H | —CH₃ |
| —OEt | —Cl | —CH₂C₆H₅ |

Substitution of an OH at the R position extends the structure activity relationship (SAR) with regards to the ethers that were made at that position.

A methyl ether version of compound 2126 showed an excellent activity. Compounds of the invention include other ether derivatives such as benzyl ether. It should be appreciated that the chiral versions of these compounds and additional ethers are within the scope of the present invention, e.g., R₂=Et, iPr, t-Bu, etc.).

Experimental data indicate that both the epoxide and the amine fragments are active. Additional hydrophobic interactions can be introduced to compounds of the invention, e.g., by using the following reaction strategy.

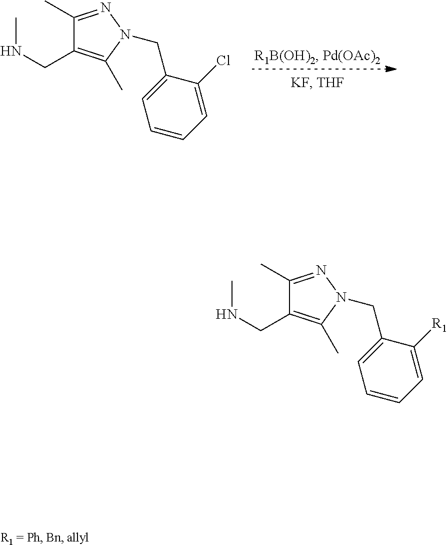

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for treating scleroderma or a related autoimmune or a fibrotic condition in a subject comprising administering a therapeutically effective amount of a toll like receptor 4 inhibitor to the subject in need of such a treatment.

2. The method of claim 1, wherein said toll like receptor 4 inhibitor is a compound of the formula: 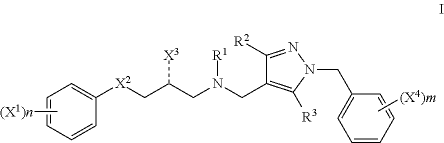
wherein
each of n and m is independently an integer from 0 to 5;
each X¹ is independently alkoxide, optionally-substituted alkyl, or alkenyl;
X² is O, NR^{a}, or S;
X³ is —OR^{b}, —SR^{b}, or —NR^{b}R^{c};
each X⁴ is independently halide or alkoxide; and
each of R^{a}, R^{b}, R^{c}, R¹, R², and R³ is independently hydrogen or alkyl.

3. The method of claim 2, wherein X² is O.

4. The method of claim 2, wherein X³ is —OH

5. The method of claim 2, wherein R¹, R² and R³ are alkyl.

6. The method of claim 5, wherein R¹, R², and R³ are methyl.

7. The method of claim 2, wherein X¹ is alkoxide, hetero-substituted alkyl or alkenyl-alkyl.

8. The method of claim 7, wherein X¹ is methoxide, methoxyethyl, or allyl.

9. The method of claim 2, wherein X⁴ is alkoxide, Cl, or F.

10. The method of claim 9, wherein X⁴ is methoxide or Cl.

11. The method of claim 1, wherein said scleroderma is a systemic sclerosis.

12. The method of claim 1, wherein said scleroderma is a diffuse scleroderma.

13. The method of claim 1, wherein said scleroderma is a limited scleroderma.

14. A method for treating scleroderma or a related autoimmune or a fibrotic condition in a subject comprising administering to the subject in need of such treatment a therapeutically effective amount of a compound of the formula: 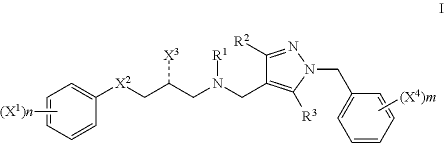
wherein
each of n and m is independently an integer from 0 to 5;
each X¹ is independently alkoxide, optionally-substituted alkyl, or alkenyl;
X² is O, NR^{a}, or S;
X³ is —OR^{b}, —SR^{b}, or —NR^{b}R^{c};
each X⁴ is independently halide or alkoxide; and
each of R^{a}, R^{b}, R^{c}, R¹, R², and R³ is independently hydrogen or alkyl.

15. The method of claim 14, wherein X² is O.

16. The method of claim 14, wherein X³ is —OH

17. The method of claim 14, wherein R¹, R² and R³ are alkyl.

18. The method of claim 17, wherein R¹, R², and R³ are methyl.

19. The method of claim 14, wherein X¹ is alkoxide, hetero-substituted alkyl or alkenyl-alkyl.

20. The method of claim 19, wherein X¹ is methoxide, methoxyethyl, or allyl.

21. The method of claim 14, wherein X⁴ is alkoxide, Cl, or F.

22. The method of claim 21, wherein X⁴ is methoxide or Cl.
